(19) United States
(12) Patent Application Publication
(10) Pub. No.: US 2014/0241511 A1
(43) Pub. Date: Aug. 28, 2014

(54) METHOD AND PROJECTION DEVICE TO MARK A SURFACE OF A 3D EXAMINATION SUBJECT

(71) Applicants: Annemarie Hausotte, Erlangen (DE); Eva Rothgang, Nuernberg (DE)

(72) Inventors: Annemarie Hausotte, Erlangen (DE); Eva Rothgang, Nuernberg (DE)

(21) Appl. No.: 14/179,874

(22) Filed: Feb. 13, 2014

(30) Foreign Application Priority Data
Feb. 28, 2013 (DE) 102013203399.0

(51) Int. Cl. A61B 6/08

(52) U.S. Cl. CPC A61B 6/08
USPC 378/206

(57) ABSTRACT

In a method and projection device to mark a surface of a three-dimensional examination subject, relief data (RD) of the surface are acquired and are used to establish a measurement information marking. A reference position value is determined, which represents a position of a radiation device of the projection device relative to the surface. A calculation of pre-distortion of the established measurement information is calculated in a processor marking depending on the relief data and the reference position value. A visually perceptible pre-distorted measurement information marking is radiated from the radiation device in the direction of the surface.

### BACKGROUND OF THE INVENTION

1. Field of the Invention

The present invention concerns a method to mark a surface of a three-dimensional examination subject, in particular the body surface of a patient. Moreover, the invention concerns a projection device to implement such a method. The invention also concerns a medical technology imaging system with such a projection device.

2. Description of the Prior Art

The determination of distances between defined points on three-dimensional bodies requires an increased effort and cost for bodies with irregular (i.e. uneven and/or flexible) surfaces. For human or animal bodies, dimensions of the body surface can vary due to (for example) breathing or peristaltic movements, or due to pressure on specific parts of the surface. For percutaneous procedures on a body, for example with a needle or a scalpel, it can be particularly important to precisely determine dimensions on the body surface, i.e. on the skin. A determination of a distance starting from a reference or start point is therefore frequently implemented manually with a flexible measurement tape that can be adapted to a certain degree to the surface geometry (topography) of a body. The use of such a measurement tape can have a number of disadvantages for the operator and/or the patient, for example in the case of surgical procedures on a patient. Among these disadvantages are, for example, an increased effort to ensure the sterility of the measurement tape, a more difficult handling of the tape at some parts of the body, the risk of the measurement tape slipping, and additional individual manual errors that can lead to measurement errors.

### BRIEF SUMMARY OF THE INVENTION

In view of the problem described above, an object of the invention is to provide a method for marking a surface with measurement information more simply and precisely.

This object is achieved by a method according to the invention for marking a surface of a three-dimensional examination subject with a projection device that includes at least the following steps:
acquire relief data of the surface,
establish or define a measurement information marking,
determine a reference position that represents a position of a radiation device of the projection device relative to the surface,
calculate a pre-distortion of the established measurement information marking depending on the relief data and the reference position value,
radiate, humanly pre-distorted visible measurement information marking created from the pre-distortion of the measurement information marking, in the direction of the surface.

A measurement information marking” as used herein means a marking that includes information about specific, relevant measurements, such as defined measurement distances, a position and/or extent of defined possible target areas, structures, etc. In particular, this can thus be a measurement information pattern composed of marking points and/or lines, wherein the individual marking points and/or lines are situated at defined distances from one another and thereby serve as measurement information for an operator. The measurement pattern can also be a depiction that represents (for example) an internal anatomical structure (such as a specific organ or multiple organs) of the examination subject. The measurement information marking can advantageously be a raster, grid or lattice that is formed from the points and/or the lines that (for example) are displayed on the surface by means of colored beams of light. The “mapping” of the measurement information marking can include a selection of stored types of markings (for example measurement grid or defined organ), as well as possibly a determination of a unit of measure (for example a metric or non-metric unit of measure) and a measurement size (for example 5 mm or 1 cm or 2 cm), for example as a distance between two intersection points of a measurement grid.

The radiation device of the projection device can be at least one light source that projects visible light (for example red or green light) as a marking medium onto the surface. The examination subject can be inanimate or animate and, for example, a plant, a mineral, or a human or animal body. The surface of the examination subject can in principle be the entire surface of the subject. When marking a three-dimensional examination subject using a punctiform radiation device, the surface most often forms a partial surface of the examination subject.

The “reference position value” includes information as to what distance, at what inclination, and in what alignment the region of the surface of the examination subject onto which a marking information should be projected, is situated relative to the radiation device. For example, the distance can be a distance between the light source and a reference point on a skin of a patient.

The relief data represent a topographical profile of the surface of the examination subject, and therefore its different elevations and/or depressions relative to a reference plane that can be formed by (for example) a contact surface of the examination subject (thus for instance a patient table). As used herein, such a relief is the sum of the contour or profile segments of the examination subject relative to (for example) parallel slice planes through the examination subject. The relief data can be generated at an arbitrary point in time before the radiation of the measurement information marking, and can be transmitted from an arbitrary source to the projection device. The individual steps of the method according to the invention can be implemented in an arbitrary order, with the exception of the described dependencies in the sequence of the data generation or processing. For example, the step of establishing the measurement information marking can take place after the step of determining the reference position value. Furthermore, the method can also include intermediate steps that are not presented (or are not presented in detail) herein. If the relief data of the surface and the reference position value (for example at a reference point on the surface) are known, spatial coordinates can be calculated for any arbitrary point on the surface, and thus for every arbitrary marking point or target point (the points at which a marking takes place on which, for example, a line, a light point, an image etc. is projected), which spatial coordinates describe its position relative to the radiation device. A simple calculation of the pre-distortion can therefore take place. For a precise marking of the surface, the surface between the step of the acquiring the relief data and the step of radiating the pre-distorted measurement information marking should advantageously remain unmoved, or the movement should be detected, registered and incorporated into subsequent calculations.

The radiation of the pre-distorted measurement information marking takes place via a radiation device and results in the aligned light beams striking the surface. The calculation of the pre-distortion of the measurement information marking thus includes the step of adjusting a radiation angle, or a difference radiation angle, of the radiation device at a spatial coordinate of a target point on the surface, relative to the starting point of a light beam. This step is implemented for every individual target point.

The pre-distorted measurement information marking (corresponding to the topography of a surface) appears undistorted upon striking the surface such that—starting from a defined start point on the surface—the marking points or lines are always situated at precisely defined (preferably identical) distance measurements from one another, independent of the curvature of the surface. A distance measurement thereby corresponds to a direct path between two marking points or lines that proceeds on the uneven surface of the examination subject. The distance measurement therefore does not correspond to a distance that is measured along a virtual “line of flight” between the two marking points or lines.

The acquisition of the relief data of the surface and the radiation of the measurement information marking onto the surface preferably takes place as close to one another in time as possible. The acquisition of the relief data and the calculation of the pre-distortion can also be repeated at defined time intervals (for example in the form of a real-time measurement) in order to continuously update the measurement information marking in the case of a living, moving body. Adulterating influences of body movements on the visual appearance of a measurement grid or image, for example, can thereby advantageously be minimized, and the precision of the display can be markedly increased.

Compared to conventional methods, the method according to the invention provides the advantage that the projection of a measurement information marking onto the surface of the examination subject takes into account unevennesses of the surface. The method thereby produces a reliably precise and reproducible measurement of distances based on previously defined reference or starting points on the surface. The method leads to a more precise localization of target points on the surface. For example, these target points can be locations at which percutaneous procedures are conducted in a body, for example points at which needles, tubes or scalpels etc. are directed through the skin of a patient, as the surface of the examination subject. The use of light as a marking medium has the advantage that no sterilization and no subsequent cleaning of the surface are necessary. The inventive method avoids the need for inscribed markings, which may become smeared, and the marking medium does not occlude the surface but rather is transparent. The method according to the invention can potentially prevent serious operating errors as they can occur with a manual determination of measurements by an operator. If desired, however, a transfer of the visual measurement information marking projected onto the skin of a patient to a marking made with a pencil or with paste-like ink can take place simply and very precisely, if a permanent marking is necessary.

The invention moreover includes a projection device that is designed to implement the method according to the invention and has the following components:
a detection unit to acquire relief data of a surface of an examination subject,
an establishing unit to establish a measurement information marking,
a reference position value determination unit to determine a reference position value of a radiation device relative to the surface,
a distortion calculation unit to calculate a pre-distortion of the measurement information marking depending on the relief data and the reference position value,
a radiation device to radiate the pre-distorted measurement information marking in the direction of the surface.

The described components (in particular the establishing unit, the reference position value determination unit and the distortion calculation unit) can for the most part be designed as separate electronic units and/or as software modules, for example in a control device of a CT system. A realization of the components largely in software has the advantage that existing computed tomography systems can be retrofitted simply via a software update in order to operate in the manner according to the invention.

The invention moreover concerns a medical technology imaging system with a projection device as described above. For example, the imaging system can be a computed tomography system, a magnetic resonance tomography system, a positron emission tomography system, or a single photon emission computed tomography system.

According to an embodiment of the invention, at least one part of the relief data is generated from topometric data of the surface of the examination subject. The term “topometry” designates a measurement of a figure or shape of a surface. The topometric data can be generated according to known measurement methods (based on triangulation, for example) that measure (detect) the shape of surfaces with high resolution. For example, one possible method for this is described in Zhang, Song/Huang, Peisen S.: High-resolution, real-time three-dimensional shape measurement, in: Optical Engineering vol. 45 no. 12 (2006), 123601_{—}1-8.

According to a further embodiment of the method according to the invention, at least a portion of the relief data is generated from volume image data and/or slice image data and/or projection image data of the examination subject. This includes the possibility of the relief data being calculated exclusively from volume image data and/or slice image data and/or projection image data of the examination subject. The volume image data and/or slice image data and/or projection image data can be acquired using an arbitrary imaging system (for example a computed tomography system) to acquire the inside of a three-dimensional body. The data can subsequently be fed into a computer of a projection device via an interface. For an examination of a human body with a defined finding of interest, volume image data and/or slice image data and/or projection image data of a body segment or of an entire body are frequently generated anyway. The embodiment according to the invention thus offers the advantage that a separate method step of a measurement of a topographical profile of a surface of the examination subject can be spared in that an already present set of volume image data and/or slice image data and/or projection image data is used to calculate the relief data.

According to an alternative or additional embodiment, at least a portion of the relief data can be generated from image exposures of the outside of the examination subject. The relief data can also be calculated exclusively from such image exposures. The image exposures can include arbitrary images of an external (i.e. visible) surface of the examination subject. For example, the exposures can show two-dimensional images, wherein distance data that represent a distance from the acquisition point are stored for each individual image point (or pixel). This embodiment is cost-effective and saves an unwanted exposure of a patient via x-ray radiation (as is incurred in radiography), for example. It has additionally proven to be realizable in a simple manner.

The image exposures of the examination subject advantageously include stereoscopic image exposures. Two two-dimensional image exposures of the surface of the examination subject are thereby generated with a spatial offset. Information about a three-dimensional extent of the examination subject can be obtained from the comparison of the image exposures with the incorporation of the offset. One possible method for this is described by Ahlvers, Udo/Zölzer, Udo/Heinrich, Gerd: Adaptive Coding, Reconstruction and 3D Visualisation of Stereoscopic Image Data, Proceedings of the 4th IASTED International Conference on Visualisation, Imaging, and Image Processing (VIIP'04), Marbella, Spain, Sep. 6-8, 2004. This embodiment also has the advantage of being implemental without a radiation exposure of the examination subject.

In a preferred variant, a marking of the examination subject can have a nonspecific measurement information marking (for example a measurement grid to indicate distances on the surface) and/or the specific measurement information marking. This embodiment offers the advantage that numerous items of measurement information can be shown on the surface of the examination subject, such that the use spectrum of the method according to the invention is markedly expanded.

Additionally or alternatively, the measurement information marking includes a patient-specific measurement information marking. The patient-specific measurement information marking can include individual measurement information of a concrete examination subject that, for example, represent physiological characteristics on its surface or inside it.

The patient-specific measurement information marking is advantageously based on image data of the inside of the examination subject. For example, it can include a detailed slice or projection image of an organ that was essentially generated in a plane parallel to the surface of the examination subject on which the projection takes place. It can additionally or alternatively include a contour of the organ and/or interior structure in the organ. For example, information about blood vessels, bones, heart or liver can therefore be projected onto the skin of a patient such that they precisely reflect the actual position of the respectively depicted internal organ in a plan view on the patient. This method achieves a significant increase in the safety for the patient for implementation of subsequent intracorporeal procedures on the basis of the marking: Due to its precision, the method causes as little damage as possible to the body tissue.

According to a further preferred embodiment, the radiation device is a laser system. This offers the advantage of a particularly precise marking of a surface of the examination subject. Moreover, a laser beam can not only mark a point and/or a line on the surface, but also can provide a designation of the direction in which a defined point inside or below the surface of the examination subject can be reached. Penetration into the examination subject at a defined angle can prove to be very advantageous, for example for drilling or cutting through the surface by means of a probe, an endoscope, a drill or scalpel; defined (for example sensitive) areas inside the examination subject can be bypassed, for example by the procedure being implemented at an angled direction relative to the surface instead of an orthogonal direction. The laser system can include a deflection unit, for example a mirror system and/or a prism system that deflects the laser beam in various directions and therefore can cover larger projection areas. The laser system can also include multiple lasers and/or deflection units, for example in order to project specific types of measurement information markings onto a surface with laser beams of different colors. For example, a marking grid with blue light can be blended onto the skin of a patient, and the contours of a kidney of the patient can be depicted as an overlay with green light. The optimal positioning of an endoscope or a puncture [fine] needle could then be marked with a red light beam.

### BRIEF DESCRIPTION OF THE DRAWINGS

FIG. 1 schematically illustrates a marking method of a radiation device on a straight surface.

FIG. 2 schematically illustrates a marking method of a radiation device as in FIG. 1 on a curved surface.

FIG. 3 schematically illustrates a marking method of a radiation device as in FIG. 1 on a curved surface with a pre-distortion according to the invention.

FIG. 4 schematically illustrates a computed tomography system with a laser system to implement the marking method according to FIG. 3.

FIG. 5 shows a variant of a marking method according to the invention at a three-dimensional body.

### DESCRIPTION OF THE PREFERRED EMBODIMENTS

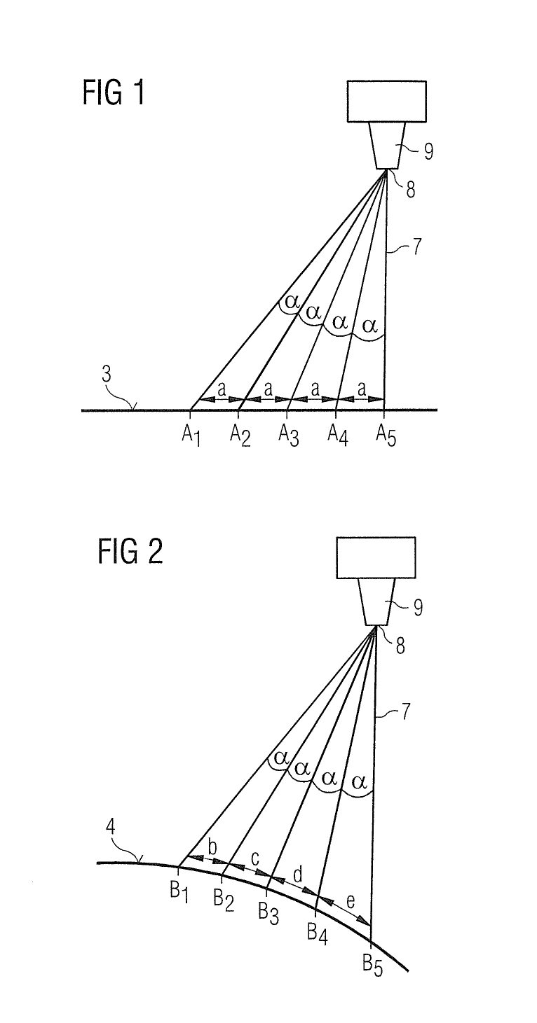

FIG. 1 shows a laser system 9 with a deflection unit 8 (as a radiation unit) and a planar surface 3 that are facing towards one another. The laser system 9 emits fan-shaped light beams 7 in the direction of the surface 3. An arbitrary light beam 7 and a respective next closest light beam 7 are always situated at an identical difference radiation angle a relative to one another. The planar property of the surface 3 and the identical difference radiation angle a of the adjacent light beams 7 lead to the situation that the light beams 7 strike at points A₁, A₂, A₃, A₄, A₅ on the surface 3, of which each point A₁, A₂, A₃, A₄, A₅ lies at an identical distance a from a respective adjacent point. This thus means that a path a between the point A₁ and the point A₂ is just as long as a path a between the point A₂ and the point A₃, or between the point A₃ and the point A₄ or between the point A₄ and the point A₅. A correct, uniform spacing or, respectively, measurement grid 5 can be shown with this method.

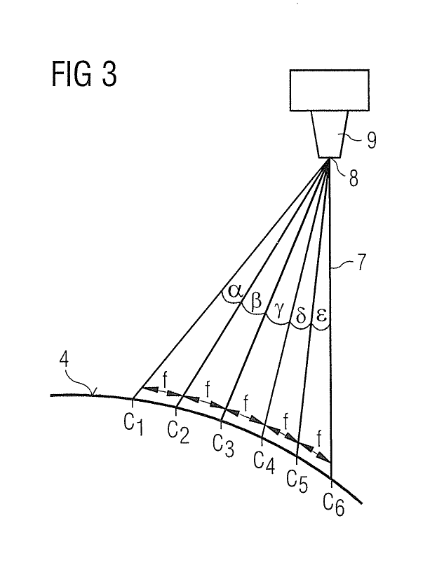

FIG. 2 differs from FIG. 1 in that the laser system 9 emits the light beams 7 in the direction of a curved surface 6. The light beams 7 strike at points B₁, B₂, B₃, B₄, B₅ on the curved surface 6. The curvature of the surface 6 now has the effect that the intervals of the light beams 7 striking the surface 6 are nevertheless different given an identical difference radiation angle a (as in FIG. 1) between two adjacent, arbitrary light beams 7. This means that a distance b between the adjacently situated points B₁ and B₂ and a distance c between the adjacently situated points B₂ and B₃ are of different lengths. The distances b, c, d, e are thereby understood as paths between the corresponding points B₁ and B₂, B₂ and B₃, B₃ and B₄ and B₄ and B₅ that are measured as they follow the curvature course (i.e. on the surface 6). The concrete position of the curved surface 6 relative to the deflection unit 8 and the shown curved course have the effect that the distance c is greater than the distance b, the distance d is greater than the distance c and the distance e is greater than the distance d. Without a variation of the difference radiation angle, the uniform spacing or, respectively, measurement grid 5 on a planar surface (see FIG. 1) is thus non-uniform or, respectively, “skewed” due to the curvature.

In order to avoid this skewing, in the marking method according to the invention shown in FIG. 3, the laser system 9 emits the light beams 7 at individual radiation angles (thus suitably “pre-distorted”) in the direction of a curved surface 6. It is controlled such that the difference radiation angles α, β, γ, δ, ε are respectively different between an arbitrary light beam 7 and a respective next closest light beam 7. The different difference radiation angles α, β, γ, δ, ε are thereby selected precisely so that distances f are identical between respective adjacent points C₁ and C₂, C₂ and C₃, C₃ and C₄, C₄ and C₅ and C₅ and C₆ at which the light beams 7 strike the surface 6. It also applies here that the distances f are understood as paths between the corresponding points C₁ and C₂, C₂ and C₃, C₃ and C₄, C₄ and C₅ and C₅ and C₆ that are measured along the curvature (i.e. on the surface 6). The measurement grid 5 pre-distorted at the point in time of the radiation is thus again visibly “deskewed” by an operator on the curved surface 6. The shown method thus offers the advantage that the measurement grid 5 projected onto the surface 6 always indicates uniform measurement intervals (set by the operator in advance), independent of the contour or relief of the surface 6.

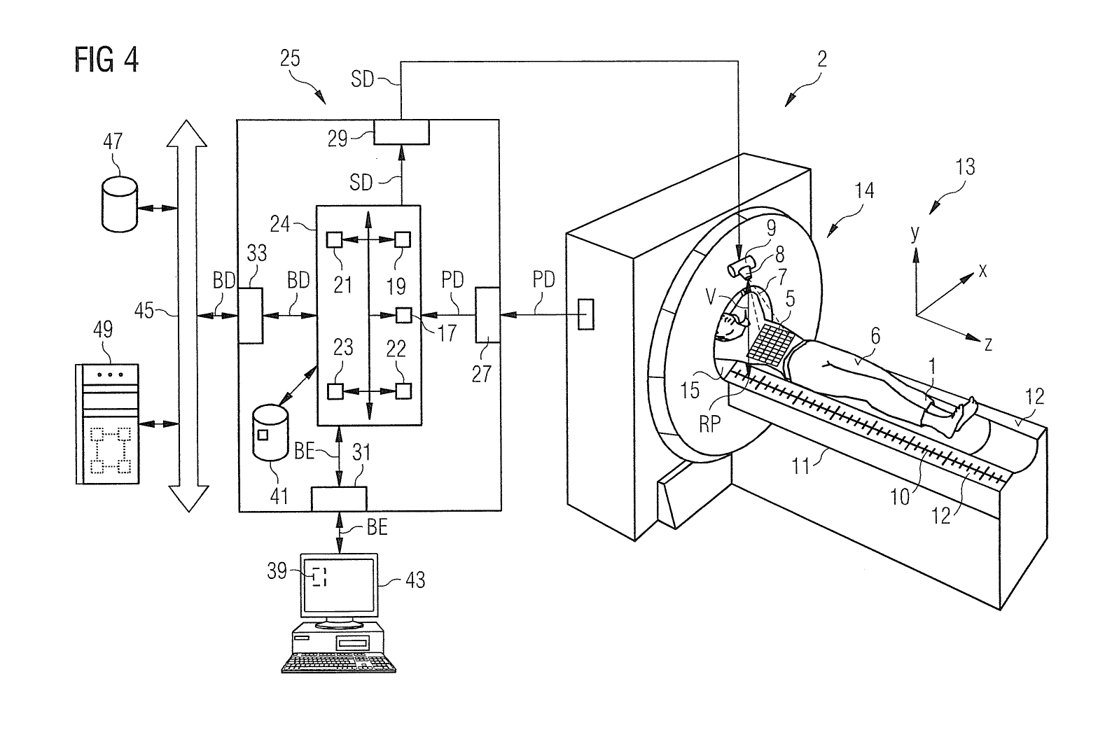

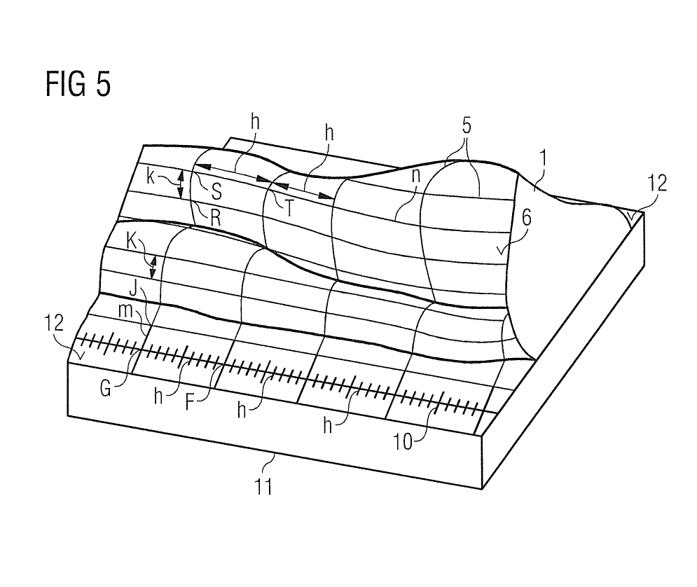

FIG. 4 shows a medical technology imaging system 13 with a projection device 2 with which the method according to the invention can be implemented.

As an example here, the medical technology imaging system 13 is a computer tomography system 13 with a scanner 14. The scanner 14 is connected in a typical manner with an electronic control system 25 that forms a component of the CT system 13 and controls the scanner in a typical manner and acquires and processes the measurement data (in particular can reconstruct image data). The scanner 14 has a patient table 11 and a measurement space 15 around which a gantry is arranged, in the shape of a ring (not shown) mounted such that it can rotate in the scanner housing, with an x-ray source (not shown) and a detector arrangement (not shown).

Here the patient table 11 can be driven into the measurement space 15. Alternatively, it is also possible to move the scanner 14 together with its housing in the direction of the patient table 11. The body 1 of a patient is borne on the patient table 11 as an examination subject. In the operation of the CT system 13, an x-ray fan or conical x-ray beam (not shown) emanating from the x-ray source propagates through the measurement space 15 in order to generate projection data PD of the body 1 from which image data BD of the inside of the body 1 can then be reconstructed in a known manner.

A radiation device 8, 9 of the projection device 2 sits on an external side of the housing of the scanner 14 above an opening of the measurement space 15. This radiation device 8, 9 comprises a laser system 9 and a deflection unit 8. The laser system 9 emits colored laser beams 7 that are deflected as controlled in defined directions by means of an adjustable mirror system. Here a uniform marking grid 5 with lines in a fixed spacing (for example a respective 1 cm interval) can—as a measurement information marking—be projected in the direction of the underlying patient table 11 onto a surface 6 of the body 1, wherein the laser beams 7 (or one laser beam with high frequency) scans across an imaginary surface parallel to the table surface (thus here in the x- and y-direction) so that (taking into account the lag [inertia] of the eye of the observer) a complete image or, respectively, the desired pattern is created. The shape and a possible pre-distortion of a marking grid 5 are thus achieved by an adjustment of the deflection unit 8. A color of the light beams 7 can be set at the laser system 9. The laser system 9, the deflection unit 8, the parts of the control system 25 that are associated with these and a controlling means 39 operable by the operator at a terminal 43 (which can be realized as a software program at a graphical user interface of the terminal, for example) together form the projection device 2.

The control system 25 of the scanner 14 controls not only the scanner 14 in the typical manner but rather, as noted, also the radiation device 8, 9 (i.e. the laser system 9 and the deflection unit 8). Therefore, only those elements or units of the control system 25 are shown that are relevant to an implementation of the individual steps of the marking method according to the invention by means of the radiation device 8, 9.

For this purpose, the control system 25 has a central control device 24 in a processor, and a scan protocol memory 41 connected with the control device 24. The control device 24 has an image generation unit 17, an establishing unit 19, a reference position value determination unit 21, a distortion calculation unit 22 and a control unit 23. The units are connected among one another via interfaces that can also be realized as software interfaces. Furthermore, the control system 25 has input and output interfaces 27, 29, 31, 33. The establishing unit 19 receives operator input signals BE from the terminal 43 via the input interface 31. The image generation unit 17 receives x-ray projection data PD from the scanner 14 via the input interface 27. The control unit 23 emits control data SD as an output to the deflection unit 8.

In the interaction with an operator, selection and control information can be entered and output via the terminal 43. For example, an operator at the terminal 43 can adjust parameters of the marking grid 5 (for example intervals between intersection points of the line raster or a presentation by means of points and/or lines) via a control window 39. A corresponding operator input signal BE is further relayed into the control device 24 or to the establishing unit 19 via the input interface 31.

The image generation unit 17 receives the x-ray projection data PD of the body 1 that are generated by the scanner 14, generates image data BD from these and extracts relief data RF from the image data BD. To reconstruct the image data BD from the x-ray projection data PD, the image generation unit 17 can also access a typical reconstruction unit (not shown) of the imaging system 13 or of the control device 24. The relief data RF represent a topographical profile of the surface 6 of the body 1 and additionally describe their position relative to a surface 12 of the patient table 11.

The reference position value determination unit 21 determines a “variable” position of the surface 6 of the body 1 relative to a fixed position of an exit point of the light beams 7 from the deflection unit 8 in a spatial coordinate system. The position of the surface 6 is inasmuch variable here since the patient table 11 with the body 1 is designed so as to be displaceable relative to the scanner 14. In contrast to this, the position of the deflection unit 8 is fixed since it is mounted permanently on the scanner 14. For this purpose, the reference position value determination unit 21 initially determines a calibration distance v as a reference position value relative to a reference point RP based on a current feed position of the patient table 11 (and therefore of the reference point RP arranged thereupon and the body 1 onto which the projection should take place) and the known position of the deflection unit 8. It furthermore then calculates the position of every point of the surface 6 on the basis of the calibration distance v and on the basis of the relief data RD. The feed position of the patient table 11 or of the body 1 thus can be determined independently by the CT system 13, or by the reference position value determination unit 21. This method step forms the requirement of a correct calculation of an alignment of the light beams 7 emitted by the deflection unit 8 at every single target point of a marking grid 5 that is projected onto the surface 6 in a subsequently step.

As explained above with FIGS. 2 and 3, the distortion calculation unit 22 calculates a pre-distortion of the marking grid 5 depending on the relief data RF and the position of the surface 6 of the body 1 as well as on the position of the deflection unit 8.

The control unit 23 generates control signals SD to control the laser system 9 or, respectively, the deflection unit 8 on the basis of computation result data of the distortion calculation unit 22. The control signals or control data SD are relayed via the output interface 29 to the laser system 9 or to the deflection unit 8. The control system 25 is linked via an output interface 33 with a bus 45 to which a mass storage 47 and a radiological information and imaging system 49 are connected. For example, image data BD, image processing commands and additional information that should be supplied for a post-processing, storage or relaying to additional image data users can be relayed via the output interface 33. The radiological information and imaging system 49 can thus execute (partial) functions of the image generation unit 17. In different intermediate steps of the method according to the invention, data sets can be cached in the mass storage 47 and then be newly supplied to the processing chain via a data processing unit.

The CT system 13 according to the invention enables that an acquisition of projection data PD or, respectively, image data BD of the body 1 can be directly assessed with a defined cognitive interest for the precise marking of defined points on the surface 6 of the body 1. This has proven to be advantageous when procedures in the body 1 should be conducted at the points, for example. An existing CT system 13 must merely be extended by the laser system 9 and a modification of the control system 25.

Only selected components of the CT system 13 (and the control system 25 included therein) that are particularly suited to clarifying the invention are shown in FIG. 4. Naturally, both devices additionally comprise a plurality of additional functional components.

FIG. 5 shows an exemplary embodiment of the principle described in FIG. 3, with different difference radiation angles (not shown) of adjacent light beams 7 emanating from a deflection unit (not shown) to mark identical measurement intervals on a curved surface 6. An arbitrary test body is shown as a three-dimensional body 1. It has an irregularly curved surface 6 and is borne on the patient table 11, which has planar lateral surfaces 12.

A marking grid 5 is projected onto the surfaces 6, 12 by means of the light beams 7. It comprises two line sets, of which the marking lines m of a first line set and marking lines n of a second line set are respectively, taken individually, exclusively situated parallel to one another on the completely flat surface 12 of the patient table 11. On this surface 12, the first set of marking lines m normally also stands at an exact right angle to the second set of marking lines n, such that the sets of marking lines m, n intersect one another at right angles.

In contrast to this, in a projection of the marking grid 5 onto the irregularly curved surface 6 right angles and straight marking lines m, n do not arise at many intersection points. However, the difference radiation angles (not shown) of adjacent light beams 7 relative to one another are selected such that a measurement interval k between intersection points G and J on the surface 12 and a measurement interval k between intersection points R and S on the surface 6 are always identical. A measurement interval h between intersection points F and G on the surface 12 and a measurement interval h between intersection points S and T on the surface 6 are similarly always identical. The shape of the marking grid 5 is thus adapted to the topography of the surface 6 such that predetermined measurement intervals are reliably reproduced or, respectively, marked by the operator.

A measurement of intervals using the marking grid 5 modified in such a manner achieves the same effect as if a flexible measuring tape is placed on the surface 6 and intervals were measured along its topographical profile. However, it has the great advantage that it is precisely reproducible at any time. However, it should thereby be ensured that the body 1 does not move between a generation of the x-ray projection data PD or, respectively, the relief data RF, a marking with the aid of the marking grid 5 and a procedure in said body 1.

A measuring stick 10 that indicates defined measurement intervals is arranged on the surface 12. It can serve to determine a feed position of the patient table 11 relative to the deflection unit (not shown) and/or to localize target points on the surface 6 of the body 1. Moreover, it can support the calibration of the establishing unit or, respectively, the control unit (both not shown) if an optical detection unit arranged at the deflection unit can detect measurement intervals on the measuring stick 10. It can therefore facilitate an adjustment of the deflection unit. It indicates spacing values for a completely planar surface that can be used at the deflection unit as reference values for a selection of the difference radiation angle.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to mark a surface of a three-dimensional examination subject with a projection device, comprising:
acquiring relief data representing a topology of said surface;
in a processor, establishing a measurement information marking;
providing said processor with a reference position value that designates a position of a radiation device of the projection device relative to the surface;
in said processor, calculating a pre-distortion of the established measurement information marking dependent on said relief data and said reference position value; and
providing an electronic signal from said processor to said radiation device that represents the calculated pre-distortion and, from said radiation device, radiating said measurement information marking modified by said pre-distortion calculation onto the surface to produce a visual appearance of said measurement information marking on said surface that is not distorted by said topology.

2. A method as claimed in claim 1 comprising generating at least a portion of said relief data from topometric data of said surface.

3. A method as claimed in claim 1 comprising generating at least a portion of said relief data from at least one of volume image data of the examination subject, slice image data of the examination subject, and projection image data of the examination subject.

4. A method as claimed in claim 1 comprising generating at least a portion of said relief data from image exposures of an exterior of the examination subject.

5. A method as claimed in claim 4 comprising acquiring stereoscopic image exposures of the exterior of the examination subject, as said image exposures.

6. A method as claimed in claim 1 comprising embodying patient-specific measurement information in said measurement information marking.

7. A method as claimed in claim 6 comprising obtaining said patient-specific measurement information from image data representing an interior of the examination subject.

8. A projection device comprising:
a radiation device;
a processor provided with relief data representing a topology of a surface of a three-dimensional examination subject and with a reference position value that designates a position of the radiation device relative to the surface;
said processor being configured to establish a measurement information marking;
said processor being configured to calculate a pre-distortion of the established measurement information marking dependent on said relief data and said reference position value, and to provide an electronic signal to the radiation device that represents the calculated pre-distortion; and
from said radiation device, radiating said measurement information marking modified by said pre-distortion calculation onto the surface to produce a visual appearance of said measurement information marking on said surface that is not distorted by said topology.

9. A projection device as claimed in claim 8 wherein said radiation device is a laser system.

10. A medical imaging system comprising:
a data acquisition device configured to acquire relief data representing a topology of a surface of a three-dimensional examination subject;
a radiation device;
a processor provided with said relief data representing said topology of said surface and with a reference position value that designates a position of the radiation device relative to the surface;
said processor being configured to establish a measurement information marking;
said processor being configured to calculate a pre-distortion of the established measurement information marking dependent on said relief data and said reference position value, and to provide an electronic signal to the radiation device that represents the calculated pre-distortion; and
from said radiation device, radiating said measurement information marking modified by said pre-distortion calculation onto the surface to produce a visual appearance of said measurement information marking on said surface that is not distorted by said topology.
